(19) United States
(12) Patent Application Publication
(10) Pub. No.: US 2016/0313244 A1
(43) Pub. Date: Oct. 27, 2016

(54) OPTICAL SENSING APPARATUS PROVIDED WITH LIGHT SOURCE AND LIGHT DETECTOR

(71) Applicant: Panasonic Intellectual Property Management Co., Ltd., Osaka (JP)

(72) Inventor: TERUHIRO SHIONO, Osaka (JP)

(21) Appl. No.: 15/131,921

(22) Filed: Apr. 18, 2016

(30) Foreign Application Priority Data
Apr. 27, 2015 (JP) 2015-090856

(51) Int. Cl. G01N 21/49; G01N 21/90; G01N 33/02; G01N 21/47

(52) U.S. Cl. CPC G01N 21/49; G01N 21/4738; G01N 21/909; G01N 33/02; G01N 2021/4773; G01N 2201/0612

(57) ABSTRACT

An optical sensing apparatus according to one aspect of the present disclosure includes: at least one light source that, in operation, emits light with which a target object is irradiated; and a light detector which includes a region in which at least a part of an optical axis from the at least one light source to the target object is located, and through which the light emitted from the at least one light source passes, and at least one photo receiving element that, in operation, receives reflection scattered light from an inside of the target object, the reflection scattered light being generated due to the irradiation of the target object with the light having passed through the region, and converts the reflection scattered light into an electric signal.

### BACKGROUND

1. Technical Field

The present disclosure relates to an optical sensing apparatus and an optical sensing method that are targeted for a living body, food, or the like, and more specifically relates to an optical sensing apparatus and an optical sensing method that are capable of reducing direct reflection light from a surface of a target object, and thereby excellently detecting reflection scattered light from the inside including much information.

2. Description of the Related Art

In recent years, optical sensing apparatuses have been used that irradiate target objects such as a living body and food with light, detects reflection scattered light that is reflected and scattered inside them, and thereby can obtain useful information on those target objects in a non-contact or non-invasive manner.

When the target object is a living body, the projected light enters the inside of the living body through the skin. Reflection scattered light that is thereafter comes out from the skin includes living body information such as a state of blood because the light has passed through blood vessels and the like. A pulse, a blood flow, and an oxygen saturation of the living body, for example, become known by the detection of the reflection scattered light, and can be used for a medical examination or the like.

Moreover, as for food, the quality such as the degree of freshness and the sugar content can be inspected in a nondestructive manner by a method of projecting light to the food and detecting reflection scattered light from an inside of the food. In particular, the method is useful for perishable food. In supermarkets, perishable food is sold by being contained in a container with plastic wrap (transparent film) or a transparent lid in many cases. The method of projecting light to perishable food and detecting reflection scattered light from an inside of the food enables consumers to buy the perishable food while checking the state of the perishable food through the transparent lid or the plastic wrap.

### SUMMARY

In one general aspect, the techniques disclosed here feature an optical sensing apparatus including: at least one light source that, in operation, emits light with which a target object is irradiated; and a light detector which includes a region in which at least a part of an optical axis from the at least one light source to the target object is located, and through which the light emitted from the at least one light source passes, and at least one photo receiving element that, in operation, receives reflection scattered light from an inside of the target object, the reflection scattered light being generated due to the irradiation of the target object with the light having passed through the region, and converts the reflection scattered light into an electric signal.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a cross-sectional view illustrating a configuration of an optical sensing apparatus according to a first embodiment, and a situation where the optical sensing apparatus irradiates a target object with light from a light source, and detects light;

FIG. 1B is a plan view illustrating a configuration of a photo receiving element side of a light detector in the optical sensing apparatus according to the first embodiment;

FIG. 2A is a cross-sectional view illustrating light beams of incident light and direct reflection light when an optical axis of substantially parallel light that is the incident light is inclined from the normal incidence with respect to a surface of the target object in the optical sensing apparatus according to the first embodiment;

FIG. 2B is a plan view illustrating a position of a light flux of the direct reflection light to the photo receiving element side of the light detector when the optical axis of the substantially parallel light that is the incident light is inclined from the normal incidence with respect to the surface of the target object in the optical sensing apparatus according to the first embodiment;

FIG. 3 is a plan view illustrating another configuration of the photo receiving element side of the light detector in the optical sensing apparatus according to the first embodiment;

FIG. 4 is a cross-sectional view illustrating a configuration of an optical sensing apparatus according to a second embodiment, and a situation where the optical sensing apparatus irradiates a target object with light from a light source, and detects light;

FIG. 5A is a cross-sectional view illustrating, when an optical axis of substantially parallel light that is incident light and an installation angle of a transparent lid are inclined from reference values with respect to a surface of the target object, light beams of the incident light, direct reflection light from the transparent lid, and direct reflection light from the surface of the target object in the optical sensing apparatus in the second embodiment;

FIG. 5B is an explanation view illustrating, when the optical axis of the substantially parallel light that is the incident light and the installation angle of the transparent lid are inclined from reference values with respect to the surface of the target object, a position of a light flux of the direct reflection light from the transparent lid to the light detector on the photo receiving element side, and a position of a light flux of the direct reflection light from the target object, in the optical sensing apparatus in the second embodiment;

FIG. 6A is a cross-sectional view illustrating a configuration of an optical sensing apparatus according to a third embodiment, and a situation where the optical sensing apparatus irradiates a target object with light from a light source, and detects light;

FIG. 6B is a configuration view of a light detector on the photo receiving element side in the optical sensing apparatus in the third embodiment;

FIG. 7A is a cross-sectional view illustrating a configuration of an optical sensing apparatus according to a fourth embodiment, and a situation where the optical sensing apparatus irradiates a target object with light from a light source, and detects light;

FIG. 7B is a configuration view of a light detector on the photo receiving element side in the optical sensing apparatus in the fourth embodiment;

FIG. 7C is a cross-sectional view illustrating a configuration of an optical sensing apparatus according to a fifth embodiment, and a situation where the optical sensing apparatus irradiates a target object with light from a light source, and detects light;

FIG. 7D is a configuration view of a light detector on the photo receiving element side in the optical sensing apparatus in the fifth embodiment;

FIG. 8A is a cross-sectional view illustrating a configuration of a conventional optical sensing apparatus, and a situation where the optical sensing apparatus irradiates a target object with light from a light source, and detects light;

FIG. 8B is a configuration view of a light detector on the photo receiving element side in the conventional optical sensing apparatus;

FIG. 9 is an explanation view illustrating a configuration of a conventional optical sensing apparatus of another form, and a situation where the conventional optical sensing apparatus irradiates a target object with light from a light source, and detects light; and

FIG. 10 is an explanation view illustrating a configuration of a conventional optical sensing apparatus of still another form, and a situation where the conventional optical sensing apparatus irradiates a target object with light from a light source, and detects light.

### DETAILED DESCRIPTION

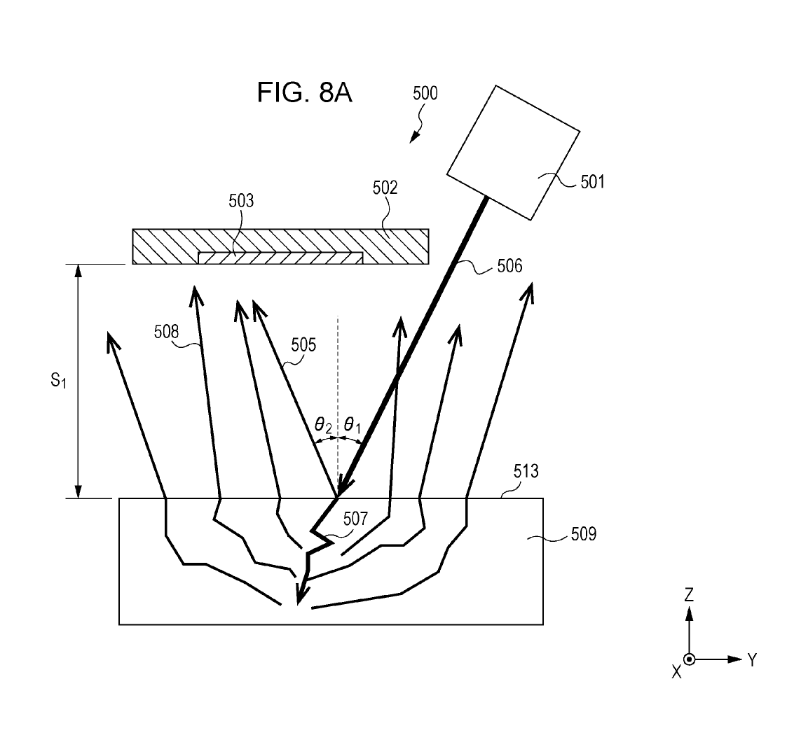

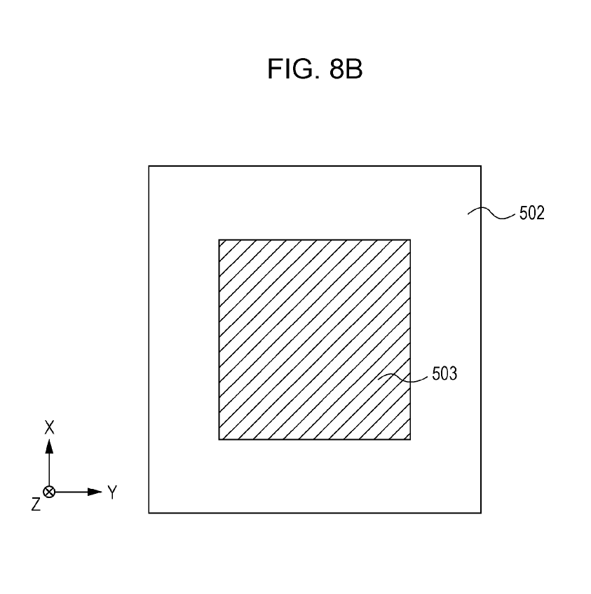

FIG. 8A is an explanation view illustrating a configuration of a conventional optical sensing apparatus, and a situation where the conventional optical sensing apparatus irradiates a target object with light from a light source, and detects light. FIG. 8B is a configuration view of a light detector on the photo receiving element side in the conventional optical sensing apparatus.

A conventional optical sensing apparatus 500 illustrated in FIGS. 8A and 8B illustrates a typical optical sensing apparatus for a target object such as a living body. In the optical sensing apparatus 500 illustrated in FIG. 8A, incident light 506 emitted from a light source 501 is incident into a target object 509 in an inclined direction at an incident angle θ₁, enters an inside thereof, and thereafter is scattered by the inside tissue to become internal scattered light 507. As a result, the optical sensing apparatus 500 has such a configuration that a photo receiving element 503 of a light detector 502 detects reflection scattered light 508 coming out from the inside.

Direct reflection light 505 reflects on a surface 513 that is a boundary surface of the target object 509 at an emission angle θ₂ (when the surface is a flat surface, θ₂=θ₁). In the configuration of the optical sensing apparatus 500, the direct reflection light 505 having a large quantity of light is mostly incident into the photo receiving element 503. This causes such a problem that a signal-to-noise ratio of the reflection scattered light 508 from the inside in which information as an original objective is included is low.

Moreover, the incident angle θ₁ close to 0 (normal incidence) of the incident light 506 to the target object 509 enables the incident light 506 to deeply enter the inside of the target object 509. To make the incident angle θ₁ close to 0, a sufficiently large distance S₁ from the photo receiving element 503 to the surface 513 of the target object 509 is required to allow the incident light 506 to enter in the inclined direction, and as a result, the size also becomes large. The larger S₁ increases the reflection scattered light 508 that does not enter the photo receiving element 503 but goes through in the transverse direction. Thus, there have been problems of the low efficiency of detecting the reflection scattered light 508 from the inside and the like.

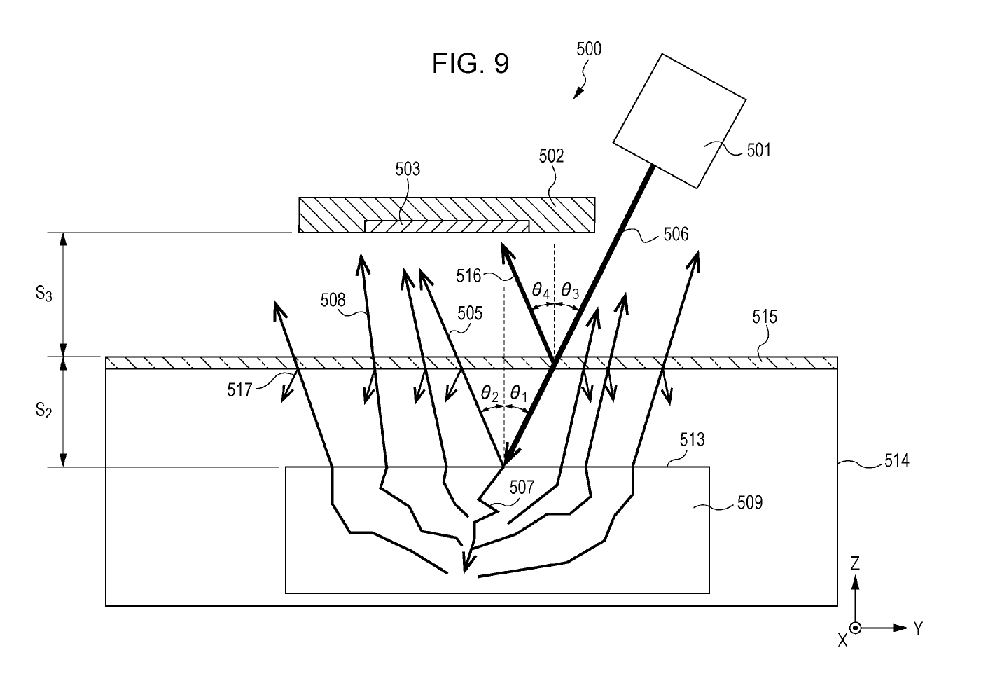

FIG. 9 is an explanation view illustrating a configuration of a conventional optical sensing apparatus of another form, and a situation where the conventional optical sensing apparatus irradiates a target object with light from a light source, and detects light.

The optical sensing apparatus in FIG. 9 illustrates a typical optical sensing apparatus for a target object that is perishable food contained in a container 514 including a transparent lid 515 such as a plastic wrap that is a transparent film. A distance from the photo receiving element 503 to the transparent lid 515 is denoted by S₃, and a distance from the transparent lid 515 to the surface 513 of the target object 509 is denoted by S₂.

The optical sensing apparatus has such a configuration that the incident light 506 from a light source 1 passes through the transparent lid 515 in the oblique direction at an incident angle θ₃. Then the incident light 506 is projected to the target object 509 in the oblique direction at the incident angle θ₁ and enters the inside of the target object 509. Thereafter, the incident light 506 is scattered by the inside tissue to become the internal scattered light 507, and as a result, the photo receiving element 503 of the light detector 502 detects the reflection scattered light 508 coming out from the inside of the target object 509.

The optical sensing apparatus 500 having such a configuration causes a problem that, in addition to the direct reflection light 505 that is reflected on the surface 513 of the target object 509 at the emission angle θ₂ (when the surface is flat, θ₂=θ₁), the quantity of light of direct reflection light 516 that is reflected on the transparent lid 515 at an emission angle θ₄ (the surface is flat in many cases, thus θ₄=θ₃) tends to become further larger than the quantity of light of the direct reflection light 505, and the direct reflection light 505 and the direct reflection light 516 are also incident into the photo receiving element 503. Thus it becomes more difficult to detect only the reflection scattered light 508 including the information from the inside, and as a result, the signal-to-noise ratio of the detection of the reflection scattered light 508 becomes worse.

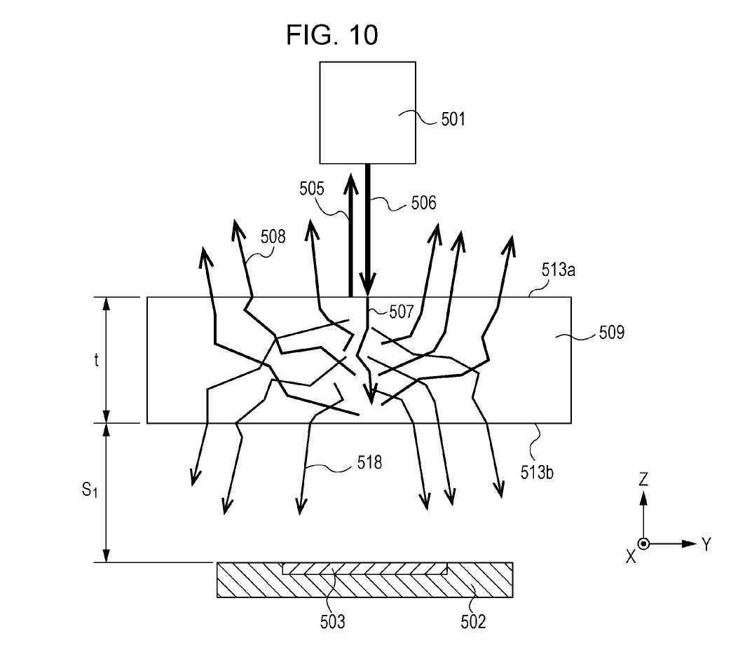

FIG. 10 is an explanation view illustrating a configuration of a conventional optical sensing apparatus of still another form, and a situation where the conventional optical sensing apparatus irradiates a target object with light from a light source, and detects light.

The optical sensing apparatus 500 illustrated in FIG. 10 illustrates a typical optical sensing apparatus of another form for a target object such as a living body. The apparatus detects transmitted scattered light 518 from the inside of the target object 509. More specifically, in the optical sensing apparatus 500, the light source 501 causes the incident light 506 to be vertically incident into the target object 509. The incident light 506 incident into the inside of the target object 509 through a surface 513a that is a boundary surface is then scattered by the inside tissue to become the internal scattered light 507. Thereafter, the internal scattered light 507 is emitted, as the transmitted scattered light 518, from the inside of the target object 509 through a rear surface 513b that is a boundary surface of the target object 509. The photo receiving element 503 of the light detector 502 that is provided at an opposite side of the target object 509 from the light source 501 then detects the transmitted scattered light 518. Here, a distance between the rear surface 513b and the photo receiving element 503 is S₁.

The optical sensing apparatus 500 having such a configuration has an advantage of an excellent detection efficiency because the direct reflection light 505 can be removed, and the distance between the target object 509 and the photo receiving element 503 of the light detector 502 can be made closer. The target object 509 having a relatively small thickness t allows the transmitted scattered light 518 to be also detected. However, the intensity of the detected light tends to be generally smaller than that of the reflection scattered light 508, thereby causing a problem of the low signal intensity.

For example, it was understood from experiments by the inventors of the present application that a transmittance T of a thumb of a person having a thickness of about t=1 cm is a comparatively small value, 1% for wavelength λ=650 nm, 0.01% for λ=532 nm, and 0.001% for λ=405 nm, and lowers as the wavelength becomes shorter within a range of visible light.

Accordingly, thin living tissues with a small thickness such as a finger and an earlobe can be measured because the transmitted scattered light 518 comes out although it is small. However, thick living tissues other than those thin living tissues cannot be measured because all the incident light is scattered and absorbed in the inside, and the signal-to-noise ratio is extremely worse. As a result, there has been a problem of a limited position suitable for the measurement.

Japanese Unexamined Patent Application Publication No. 59-150330 relates to a defect detection device that, with an objective of detecting a defect such as a scratch and dust on an information disc, makes emitted light from a laser light source parallel by a collimator lens, makes the parallel light converge by a condensing lens, condenses the converging light onto a spot having a diameter (sub-μm up to about 1 μm) the same as the size of unevennesses corresponding to information on a surface of the information disc, and detects reflection light therefrom.

The defect detection device exhibits an effect of detecting a defect on the surface with high sensitivity in such a manner that: non-scattered light that is reflection light when no defect is present on the surface of the information disc is turned back through a light path, becomes parallel light by the condensing lens, and is received by a first light detector; scattered light from a defect when the defect is present on the surface of the information disc is received by a ring-like second light detector that is arranged around the condensing lens; and a difference between output from the first light detector and output from the second light detector is detected.

The defect detection device according to Japanese Unexamined Patent Application Publication No. 59-150330 aims to detect a fine defect on a medium surface, and thus is capable of detecting a fine defect having a size the same as that of the unevennesses corresponding to the information by condensing light to the diffraction limit of light (the size of about the wavelength). However, the defect detection device is not capable of detecting a fine defect even by receiving reflection scattered light because projection of substantially parallel light onto the information disc makes a large light spot on the surface of the disc.

Moreover, the defect detection device preferably may not receive scattered light from the inside of the information disc, which adversely causes noise, because the objective is to detect a fine defect on the surface.

The present disclosure includes an optical sensing apparatus, and an optical sensing method using the same described in the following Items.

### [Item 1]

An optical sensing apparatus comprising:

at least one light source that, in operation, emits light with which a target object is irradiated; and

a light detector which includes
a region in which at least a part of an optical axis from the at least one light source to the target object is located, and through which the light emitted from the at least one light source passes, and
at least one photo receiving element that, in operation, receives reflection scattered light from an inside of the target object, the reflection scattered light being generated due to the irradiation of the target object with the light having passed through the region, and converts the reflection scattered light into an electric signal.

### [Item 2]

The optical sensing apparatus according to Item 1, wherein the at least one photo receiving element is in contact with the region.

### [Item 3]

The optical sensing apparatus according to Item 1 or 2, wherein the at least one photo receiving element surrounds the region in plan view.

### [Item 4]

The optical sensing apparatus according to Item 1, wherein a spread angle of the light having passed through the region is within ±3° in a total angle.

### [Item 5]

The optical sensing apparatus according to any one of Item 1 to 4, wherein a collimator lens that is disposed on the optical axis.

### [Item 6]

The optical sensing apparatus according to Item 1, wherein a beam diameter of the light having passed through the region is from 200 μm to 20 mm, both inclusive.

### [Item 7]

The optical sensing apparatus according to Item 1, wherein the at least one light source comprises a first light source that, in operation, emits light of a first wavelength, and a second light source that, in operation, emits light of a second wavelength.

### [Item 8]

The optical sensing apparatus according to Item 7, wherein

in plan view,

the first light source and the second light source are disposed along a first direction, and

the region has an elliptic shape having a major axis in the first direction.

### [Item 9]

The optical sensing apparatus according to any one of Items 1 to 8, wherein

the at least one photo receiving element comprises photo receiving elements, and

the photo receiving elements are disposed in a surrounding area of the region in a circumferential direction of the region with spaces that electrically insulate the photo receiving elements from one another.

### [Item 10]

The optical sensing apparatus according to any one of Items 1 to 8, wherein

the at least one photo receiving element comprises photo receiving elements, and

the photo receiving elements are disposed in a surrounding area of the region in a radial direction of the region with a space that electrically insulates the photo receiving elements from one another.

### [Item 11]

The optical sensing apparatus according to any one of Items 1 to 6, wherein the light emitted by the at least one light source is pulse light.

### [Item 12]

The optical sensing apparatus according to any one of Items 1 to 6, further comprising a flexible substrate, wherein

the at least one light source and the light detector are disposed on the flexible substrate.

### [Item 13]

The optical sensing apparatus according to any one of Items 1 to 12 further comprising a processor, wherein, in operation,

the processor performs computation of the electric signal to obtain information on the target object. The processor may include processing circuitry and a memory storing a program, wherein the program, when executed by the processing circuitry, causes the processor to perform computation of the electric signal to obtain information on the target object.

### [Item 14]

The optical sensing apparatus according to any one of Items 1 to 6, wherein 50% or more of direct reflection light that is generated due to the light having passed through the region being reflected on a surface of the target object passes through the region.

### [Item 15]

The optical sensing apparatus according to any one of Items 1 to 6, wherein the at least one light source irradiates the target object with the light, when a size of the region is denoted by d₁, and a distance from a surface of the photo receiving element opposed to the target object to a center of a light flux of the light on the surface of the target object is denoted by S₁ such that an incident angle θ₁ of the light to the target object satisfies

θ₁≦tan⁻¹(d₁/(4S₁)).

### [Item 16]

The optical sensing apparatus according to Item 15, wherein when a spot diameter of direct reflection light that is generated due to the light reflected on the surface of the target object, on a surface of the photo receiving element is denoted by w, the incident angle θ₁ satisfies

θ₁≦tan⁻¹((d₁−w)/(4S₁)).

### [Item 17]

The optical sensing apparatus according to any one of Items 1 to 6, wherein

the target object is disposed inside a container including a transparent lid, and

50% or more of direct reflection light that is generated due to the light reflected on a surface of the target object and a surface of the transparent lid passes through the region.

### [Item 18]

The optical sensing apparatus according to any one of Items 1 to 6, wherein

the target object is disposed inside a container including a transparent lid, and

the at least one light source irradiates the target object with the light, when a size of the region is denoted by d₁, a distance from a surface of the photo receiving element opposed to the target object to a center of a light flux of the light on a surface of the transparent lid is denoted by S₃, and an inclination angle of the transparent lid with respect to the surface of the target object is denoted by θ₅ such that an incident angle θ₁ of the light to the target object satisfies

θ₁≦tan⁻¹(d₁/(4S₃))−θ₅.

### [Item 19]

The optical sensing apparatus according to Item 18, wherein when a spot diameter of direct reflection light that is generated due to the light reflected on the surface of the transparent lid, on the surface of the photo receiving element is denoted by w, the incident angle θ₁ satisfies

θ₁≦tan⁻¹((d₁−w)/(4S₃))−θ₅.

### [Item 20]

An optical sensing method using the optical sensing apparatus according to Item 1, comprising:

irradiating the target object with the light, when a size of the region is denoted by d₁, and a distance from a surface of the photo receiving element opposed to the target object to a center of a light flux of the light on the surface of the target object is denoted by S₁ such that an incident angle θ₁ of the light to the target object satisfies

θ₁≦tan⁻¹(d₁/(4S₁))

by the at least one light source; and

detecting the reflection scattered light by the light detector.

### [Item 21]

The optical sensing method according to Item 20, wherein when a spot diameter of direct reflection light that is generated due to the light reflected on the surface of the target object, on a surface of the photo receiving element is denoted by w, the incident angle θ₁ of the light to the target object satisfies

θ₁≦tan⁻¹((d₁−w)/(4S₁)).

### [Item 22]

An optical sensing method using the optical sensing apparatus according to Item 1, wherein

the target object is disposed inside a container including a transparent lid, and

the optical sensing method comprises: irradiating the target object with the light, when a size of the region is denoted by d₁, a distance from a surface of the photo receiving element opposed to the target object to a center of a light flux of the light on the surface of the transparent lid is denoted by S₃, and an inclination angle of the transparent lid with respect to the surface of the target object is denoted by θ₅ such that an incident angle θ₁ of the light to the target object satisfies

θ₁≦tan⁻¹(d₁/(4S₃))−θ₅

by the at least one light source; and

detecting the reflection scattered light by the light detector.

### [Item 23]

The optical sensing method according to Item 22 wherein when a spot diameter of direct reflection light that is generated due to the light reflected on the surface of the transparent lid, on a surface of the photo receiving element is denoted by w, the incident angle θ₁ of the light to the target object satisfies

θ₁≦tan⁻¹((d₁−w)/(4S₃))−θ₅.

In the following embodiments, an optical sensing apparatus and an optical sensing method that are capable of reducing the detection amount of direct reflection light from a surface of a target object or a cover that covers the target object, and detecting reflection scattered light from an inside of the target object with a higher signal-to-noise ratio will be described with reference to the drawings. Note that, each of the embodiments described below indicates one specific example of the present disclosure. Accordingly, numerical values, shapes, materials, constituent elements, layout and connection form of the constituent elements, steps, and the order of the steps indicated in the following embodiments are merely examples, and are not intended to limit the present disclosure. Therefore, among constituent elements described in the following embodiments, those constituent elements that are not described in independent claims indicating highest-level concepts of the present disclosure are described as arbitrary constituent elements.

Moreover, the drawings are schematic diagrams, and illustration thereof is not necessarily accurate. Furthermore, in the drawings, constituent elements having substantially the same configuration are assigned with the same reference numeral, and redundant description thereof is omitted or simplified.

Moreover, in the diagram illustrating the configuration of the optical sensing apparatus, incident light, direct reflection light, internal scattered light, and reflection scattered light from the inside are illustrated as light beams that progress in directions indicated by arrows, and the whole beams of each light are illustrated as a light flux. Moreover, for easy understanding, hatching of a target object to which light sensing is performed is omitted in the cross-sectional views. Moreover, the photo receiving element illustrated in the plan views is assigned with hatching that is the same as the hatching assigned to the photo receiving element illustrated in the cross-sectional views.

### First Embodiment

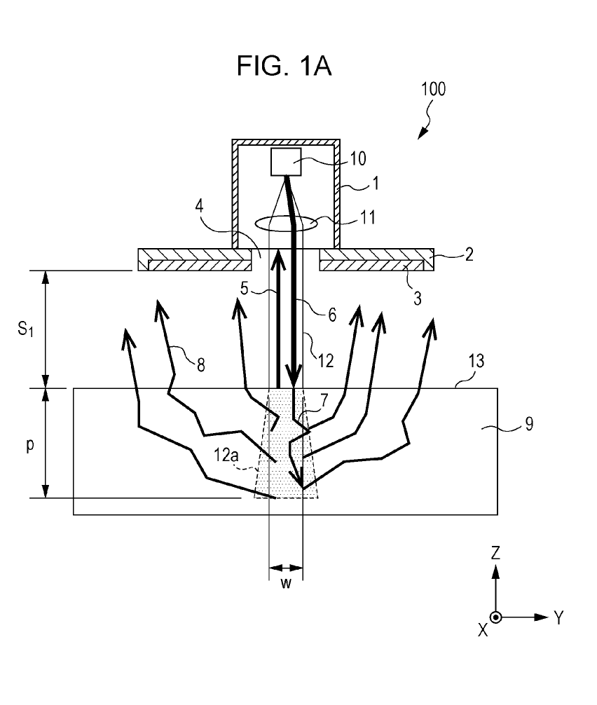

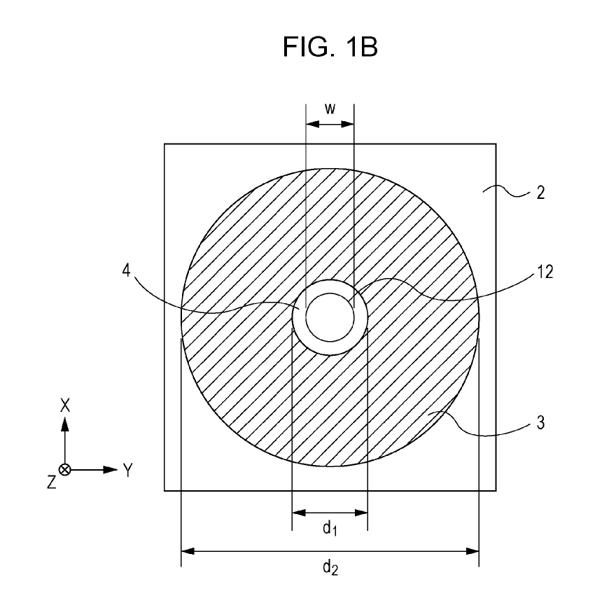

Firstly, an optical sensing apparatus and an optical sensing method according to a first embodiment will be described in details using FIG. 1A to FIG. 3. An XYZ coordinates system is assigned in the figures.

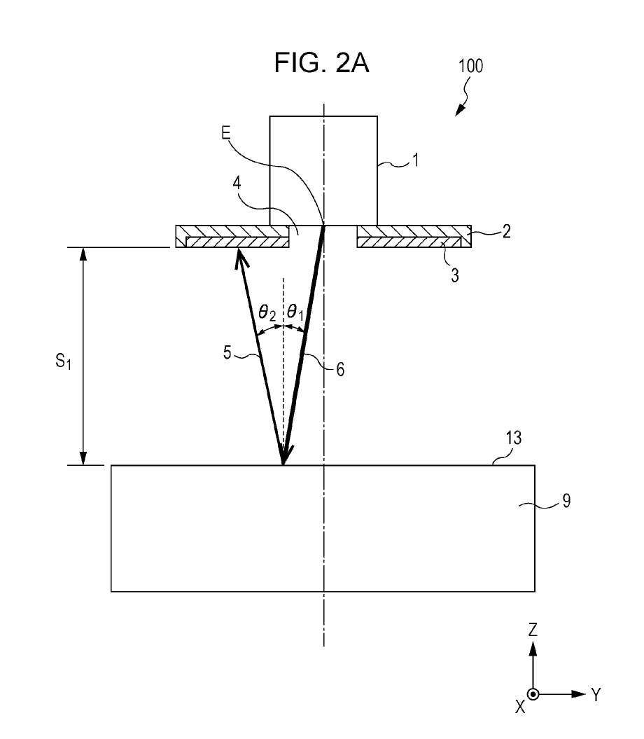

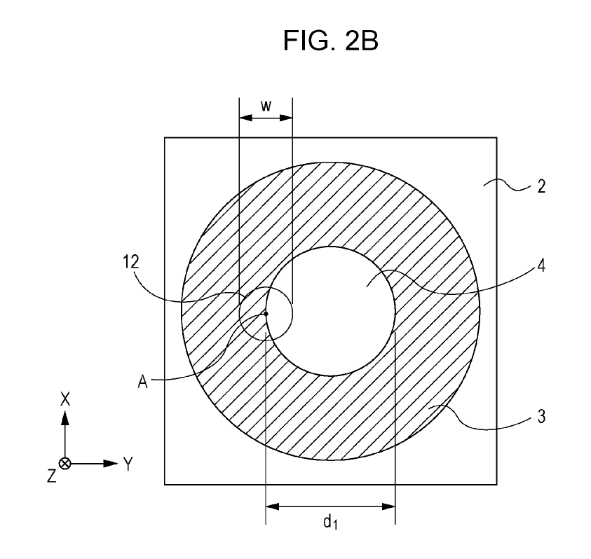

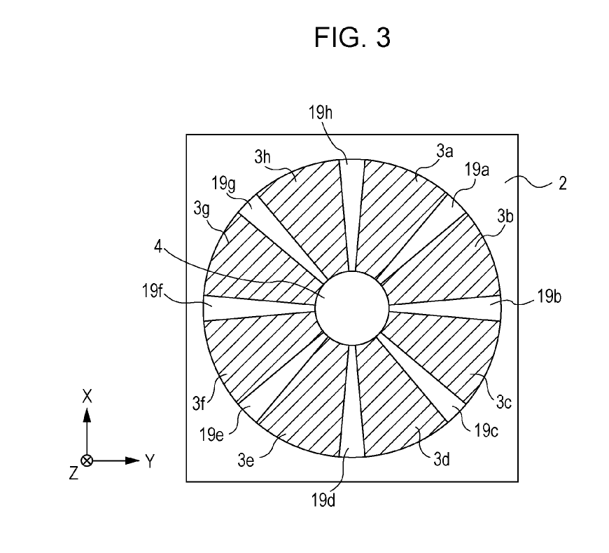

FIG. 1A is a cross-sectional view illustrating a configuration of an optical sensing apparatus according to the present embodiment, and a situation where the optical sensing apparatus irradiates a target object with light from a light source, and detects light. FIG. 1B is a plan view illustrating a configuration of a photo receiving element side of a light detector in the optical sensing apparatus according to the present embodiment. FIG. 2A is a cross-sectional view illustrating light beams of incident light and direct reflection light when an optical axis of the incident light is inclined from the normal incidence with respect to a surface of the target object in the optical sensing apparatus according to the present embodiment. FIG. 2B is a plan view illustrating a position of a light flux of the direct reflection light to the photo receiving element side of the light detector when the optical axis of the incident light is inclined from the normal incidence with respect to the surface of the target object in the optical sensing apparatus according to the present embodiment. FIG. 3 is a plan view illustrating another configuration of the photo receiving element side of the light detector in the optical sensing apparatus according to the present embodiment.

Note that, in FIG. 2A, for easy explanation, illustration of internal scattered light and reflection scattered light from the target object is omitted, and light beams indicating the progression of incident light and direct reflection light are mainly illustrated.

Note that, in the optical sensing apparatuses in the drawings, a coordinates system is determined such that an XY plane is in parallel with a plane including a surface of the target object near a measurement point of the target object, an optical axis center E at an emission end of the light source is determined to be an origin point of the XY coordinates, and a center position of the target object is set to a position where a normal from the optical axis center E intersects the target object.

An optical sensing apparatus 100 according to the present embodiment is an optical sensing apparatus that detects reflection scattered light 8 from the inside of a target object 9, and is provided with a laser chip 10 that is a light source, and a light detector 2. The optical sensing apparatus 100 according to the present embodiment is further provided with a collimator lens 11 that is disposed on an optical axis of the laser chip 10. The light detector 2 is disposed on an optical axis from the laser chip 10 to a target object. The light detector 2 includes a region 4 that includes the optical axis and through which incident light emitted from the laser chip 10 passes, and a photo receiving element 3 that receives the reflection scattered light 8 from the inside of the target object 9, the reflection scattered light 8 generated in such a manner that incident light 6 having passed through the region 4 is projected to the target object 9, and converts the reflection scattered light 8 into electric signals.

The laser chip 10 emits the incident light 6 that is projected to the target object 9. The collimator lens 11 makes the incident light 6 substantially parallel. The “substantially parallel light” refers to light having a spread angle within ±3° in the total angle, for example. The substantially parallel light will be described later. The laser chip 10 and the collimator lens 11 are stored in a housing 1.

In the present embodiment, the photo receiving element 3 is provided at a position that is opposed to the target object 9 and surrounds the region 4. As one example, the region 4 may be provided in a center of the photo receiving element 3. A placement position of the region 4 may be shifted from the center of the photo receiving element 3.

An optical sensing method according to the present embodiment is an optical sensing method that uses the optical sensing apparatus 100 in the present embodiment, and includes, when a size of the region 4 seen in a Z direction is denoted by d₁, and a distance in the Z direction from the photo receiving element 3 to a center of a light flux of the incident light 6 on a surface 13 of the target object 9 is denoted by S₁, emitting the incident light 6 towards the target object 9 by the laser chip 10 that is a light source such that an incident angle θ₁ (an angle made by a normal to the surface 13 near a measurement position of the target object 9 and the optical axis of the incident light 6) of the incident light 6 relative to the target object 9 satisfies θ₁≦tan⁻¹(d₁/(4S₁)), and detecting by the reflection scattered light 8 from the inside of the target object 9 by the light detector 2. Note that, the size of the region 4 herein indicates a diameter of the region 4 when the shape of the region 4 is circular as seen in the Z direction, a length of a minor axis for the region 4 when the region 4 is an ellipse, a diameter of an inscribed circle of the region 4 when the region 4 is a square, and a length of a minor axis of an inscribed ellipse of the region 4 when the region 4 is a rectangle. Moreover, the size of the region 4 herein indicates a diameter of an inscribed circle of the region 4, when the region 4 is a regular polygon when seen in the Z direction.

In this process, the optical sensing method may include causing the incident light 6 to be emitted towards the target object 9 such that, when a spot diameter of direct reflection light 5 obtained by the incident light 6 being reflected on the surface 13 of the target object 9, on the surface of the photo receiving element 3, is denoted by w, an incident angle θ₁ of the incident light 6 relative to the target object satisfies θ₁≦tan⁻¹((d₁−w)/(4S₁)). Such an optical sensing method further improves the signal-to-noise ratio of signals.

Note that, the “substantially parallel light” refers to light having a spread angle within ±3° in the total angle, for example. Diverging light has a plus spread angle, and converging light has a minus spread angle. The substantially parallel light may have a spread angle of within ±2 tan⁻¹[(1.41d₁−w)/(4S₁)] in the total angle. Too large spread angle of the incident light 6 results in causing most of the direct reflection light 5 from the surface of the target object 9 to reach the photo receiving element 3. Accordingly, if the spread angle that allows 50% or more of the direct reflection light 5 from the surface of the target object 9 to pass through the region 4 is considered, when the size of the region 4 of the light detector 2 is denoted by d₁, such a degree of parallelism that a spot diameter of the direct reflection light 5 that spreads on a surface of the photo receiving element 3 is √2(=1.414) times or less with respect to the size of the region 4 is necessary. When a distance from the photo receiving element 3 to the surface of the target object 9 is denoted by S₁, and a spot diameter of a light flux of the incident light on the surface of the photo receiving element 3 is denoted by w, a spread angle of the incident light 6 can be calculated within ±2 tan⁻¹[(1.41d₁−w)/(4S₁)] in the total angle. For example, in a case of S₁=10 mm and d₁=W=1 mm, the spread angle of the incident light 6 is within ±1.1°, and in a case of S₁=10 mm and d₁=w=2 mm, the spread angle of the incident light 6 is within ±2.3°.

The target object 9 in the optical sensing apparatus 100 according to the present embodiment is a living body, for example. Light having been projected to the living body as the incident light 6 enters the inside of the living body through a skin, and thereafter comes out again through the skin as the reflection scattered light 8, which includes living body information such as a state of blood because the light passes through a blood vessel and the like. Accordingly, a pulse, a blood flow, and an oxygen saturation of the living body, for example, become evident by the detection of the reflection scattered light 8. Therefore, the optical sensing apparatus 100 can be used for a medical examination or the like.

As illustrated in FIG. 1A, the optical sensing apparatus 100 according to the present embodiment is provided with the collimator lens 11 on the optical axis of the laser chip 10. Note that, the name of the collimator lens is assigned for convenience, and the collimator lens is identical with a general lens.

An emission end of the laser chip 10 is placed at an approximate focal point position of the collimator lens 11 to allow the incident light 6 having passed through the collimator lens 11 to become substantially parallel light. The position of the collimator lens 11 is moved in the optical axis direction (±Z direction) to change a substantially parallel state of the incident light 6. For example, the collimator lens 11 is moved in the +Z direction to cause the incident light 6 to become diverging light, and moreover, the collimator lens 11 is moved in the −Z direction to cause the incident light 6 to be closer to converging light.

The use of a green semiconductor laser having a wavelength of λ=532 nm, for example, as the laser chip 10 increases the modulation depth for the pulse wave detection because light having the wavelength absorbs large amounts of the oxidized hemoglobin and the deoxidized hemoglobin, and can configure the optical sensing apparatus 100 suitable for the purpose thereof. Note that, as for the light source, a light source that emits light of a wavelength in accordance with the purpose may be used. In particular, the use of a light source having a wavelength of λ=700 to 1300 nm, that is a so-called biological window, exhibits an effect of allowing the incident light to easily enter a living body deeper to some extent (for example, several ten millimeters).

Although the laser chip 10 that easily causes light to be substantially parallel light is used as the light source in the optical sensing apparatus 100 according to the present embodiment, an LED chip the light-emitting size of which is small (for example, 200 μm or less) may be used. The light-emitting size of the LED chip is generally larger than that of the laser chip, and thus the degree of parallelism of the incident light 6 is degraded. However, the optical sensing apparatus 100 according to the present embodiment can perform light sensing of the reflection scattered light 8 with such light that a spot diameter (diameter for which the amplitude of the incident light 6 is 1/e² times a median) of a light flux 12 of the incident light 6 on the surface 13 of the target object 9 satisfies, for example, 200 μm to 20 mm, because even if the degree of parallelism of the light flux 12 becomes worse, as described later, the light enters the inside of the target object 9, and thereafter the reflection scattered light 8 is generated from the inside.

The beam diameter (diameter the amplitude of which becomes 1/e² times a median) w of the light flux 12 of the incident light 6 is, for example, w=200 μm to 20 mm. The incident light 6 progresses in the −Z direction through the region 4 of the light detector 2, and is substantially vertically incident into the target object 9. As the incident angle becomes closer to a vertical angle, the light can enter the inside of the target object 9 deeper.

Here, a spot diameter of the light flux 12 of the incident light 6 on the surface 13 of the target object 9 is 200 μm to 20 mm, which is almost the same size as the beam diameter w, if the incident light 6 has a high degree of parallelism. The incident light 6 that is not as converging light converged on the surface 13, but as substantially parallel light the light flux 12 of which has a diameter of 200 μm or more is projected to the target object 9 to allow the substantially parallel light 6 to be projected to the surface 13 of the target object 9 with the energy a half or more of the original energy, even if vellus hair or terminal hair (diameter is about 10 to 100 μm) from the skin, fine dust having a diameter of a hundred and several ten micrometers or less that is hard to be recognized, or the like is attached to the surface 13 of the target object 9, for example. Note that, when the light flux 12 has a small spot diameter of less than 200 μm, it is difficult to form substantially parallel light because the light is prevented by the obstacles as the above, and tends to have large beam spread by diffraction(spread angle of diffraction pattern larger than 0.3 degree in the whole width).

Note that, when light that is converged to 1 μm or less on the surface 13 enters a place thereon, if dust or hair from the skin is present, the light does not enter the inside of the target object 9, and therefore, the measurement is impossible at that place.

Moreover, substantially parallel light having a comparatively large spot diameter of, for example, 200 μm to 20 mm is incident into the surface 13 of the target object 9 to average information in the inside of the target object 9 at least in terms of the size, so that positional variations depending on places of diagnostics information can be suppressed to some extent.

Further, if the spot diameter exceeds 20 mm, the collimator lens 11 that makes light substantially parallel becomes large in size to increase the cost, and a distance from the laser chip 10 to the collimator lens 11 becomes simultaneously longer, which results in the upsizing of the apparatus.

The incident light 6 after having entered the inside of the target object 9 is scattered by the inside tissue and becomes internal scattered light 7, and is simultaneously absorbed, so that the internal scattered light 7 that is present in a depth up to p (p is generally about several ten millimeters) is illustrated in FIG. 1A. A beam diameter of a light flux 12a of the internal scattered light 7 becomes larger because scattering becomes stronger as the light enters the inside deeper. The photo receiving element 3 of the light detector 2 detects the reflection scattered light 8 that comes out from the surface 13 as a reflection component after the internal scattering, and converts it into an electric signal.

The optical sensing apparatus 100 according to the present embodiment is further provided with a processor (not illustrated). An electric signal converted by the photo receiving element 3 is sent to the processor that is electrically connected the photo receiving element 3. The processor performs computation processing of the electric signal, thereby obtaining information on the inside of the target object 9.

For example, when the optical sensing apparatus 100 is used for a pulse measurement, the processor counts local maximum values and the like in a pulse wave having a periodic curve, and converts the counting result into a pulse rate.

Moreover, the processor may measure the degree of uniformity of periods of the pulse waves, and determine a mental condition of being concentrated and being relaxed. In this case, a concentrated state or a tensed state can be determined if the degree of uniformity of cycles is high, in other words, the cycle is constant, and a relaxed state can be determined if the degree of uniformity becomes low along with the respiration.

When the target object 9 is a living body, the smaller distance S₁ allows more of the reflection scattered light 8 spreading in the X and Y directions to be incident into the photo receiving element 3, so that the detection power of the reflection scattered light 8 becomes large. The value of the detection power varies depending on the measurement part and the wavelength, and a typical value is about 0.001 to several percent of the power of the incident light 6.

The light detector 2 includes the photo receiving element 3, and the region 4 through which the incident light 6 passes, as illustrated in FIGS. 1A and 1B.

The photo receiving element 3 is provided on a surface of the light detector 2 on the target object 9 side so as to be opposed to the target object 9, as illustrated in FIG. 1A. Moreover, the photo receiving element 3 is disposed to surround the region 4 in plan view, as illustrated in FIG. 1B. Moreover, although FIG. 1B illustrates the photo receiving element 3 as a circular shape, the photo receiving element 3 may have a polygonal shape, such as a rectangle, an ellipse, or a hexagon.

The region 4 through which the incident light 6 passes is a region where a center portion of the photo receiving element 3 is opened when the light detector 2 is seen in plan view, as illustrated in FIG. 1 B. The region 4 is formed larger than the beam diameter w of the light flux 12 of the incident light 6 by 10 percent or more as described above, for example, considering an alignment error. In other words, the size d₁ of the region 4 is, for example, d₁≧1.1w.

The photo receiving element 3 may be formed in such a manner that a silicon substrate having a structure of a P-intrinsic-N diode (PIN diode), for example, and having a size of a diameter d₂ is bonded to a glass substrate or the like. The region 4 may be formed in such a manner that a through hole having a size d₁ is opened in the center portion of the photo receiving element 3 by an abrading method with a whetstone, for example.

Moreover, the light detector 2 may be formed in such a manner that a silicon film is accumulated on a glass substrate using a plasma CVD method or the like to have a PIN diode structure, and a center portion is thereafter removed by etching processing using a lithography method. In this case, the center portion on the glass substrate corresponds to the region 4.

A position on the surface 13 where the reflection scattered light 8 comes out from the inside of the target object 9 substantially depends on the depth that light enters the target object 9, and is placed further apart from a center position on the surface 13 to which the incident light 6 is projected as the internal scattered light 7 enters deeper into the target object 9. In other words, the internal scattered light 7 comes out on the position of a ring-like shape having a radius as substantially same as the depth that the light enters, on the surface 13. Accordingly, the photo receiving element 3 is formed to have a ring-like or doughnut-like shape to surround the region 4, thereby making it possible to completely detect the reflection scattered light 8 that approximately uniformly comes out from the inside in the circumferential direction and increase the signal quantity of light. Moreover, although the circle is illustrated as an appearance shape of the photo receiving element 3, the appearance shape may be a polygonal shape, such as a rectangle, an ellipse, or a hexagon.

Moreover, the photo receiving element 3 has excellent light receiving efficiency and easy wiring because the photo receiving element 3 is connected in a ring-like shape. However, the shape of the photo receiving element 3 is not limited to this. For example, as illustrated in FIG. 3, the photo receiving element 3 may be formed in such a shape that multiple spaces 19 (more specifically, eight spaces 19a to 19h illustrated in FIG. 3) are radially provided to divide the photo receiving element 3 (more specifically, such a shape that the photo receiving element 3 is divided into the photo receiving elements 3a to 3h illustrated in FIG. 3). In other words, the photo receiving element 3 may include the multiple photo receiving elements 3a to 3h that are divided in the circumferential direction in the surroundings of the region 4. Moreover, in this case, the light detector 2 may include the spaces 19a to 19h between the photo receiving elements 3a to 3h. The photo receiving elements 3a to 3h are electrically isolated by the spaces 19a to 19h.

With this configuration, the total light-receiving area of the photo receiving elements 3a to 3h is less than the light-receiving area of the photo receiving element 3 that is not divided into multiple regions. However, the photo receiving elements 3a to 3h are individually connected to the processor, and the light receiving amount in each of the photo receiving elements 3a to 3h is converted into an electric signal to allow inside information to be obtained. This produces an effect that inside information on the target object 9 can be detected at each place where each of the photo receiving elements 3a to 3h is disposed. The greater the divided number is, the more finely the positional information can be detected.

Meanwhile, a reflectance at the surface 13 of the target object that reflects the direct reflection light 5 depends on the refractive index thereof, and in a case of a living body, the refractive index of the skin is n=1.4 to 1.6, and thus the reflectance is about 3 to 5%. The direct reflection light 5 turns back on the surface 13 and progresses in the Z direction, passes through the region 4, and progresses in the direction of the laser chip 10, and is hard to enter the photo receiving element 3.

Further, when the surface 13 is a skin or the like and has some of unevennesses such as a fingerprint, reflection components in the inclined direction may be generated in some degree in the direct reflection light 5. For example, the optical sensing apparatus 100 may be configured such that 50% or more of the direct reflection light 5 passes through the region 4. This can suppress the quantity of noise light to the half or less to raise the signal-to-noise ratio to two times or more.

For example, it is possible to raise the rate of the direct reflection light 5 that passes through the region 4 in the Z direction by methods including a method of making the size of the region 4 (diameter) larger or making the distance S₁ from the photo receiving element 3 to the surface 13 of the target object 9 be smaller.

Next, a case where the optical axis of the incident light 6 is inclined with respect to the surface of the target object 9 in the optical sensing apparatus 100 according to the present embodiment will be described.

As illustrated in FIG. 2A, the incident light 6 enters the surface 13 of the target object 9 at an incident angle θ₁, and the direct reflection light 5 from the surface 13 is reflected at an emission angle θ₂ and is projected to a lower surface of the light detector 2. When the surface 13 is flat, θ₁=θ₂ is obtained. If the surface 13 has roughness, the emission angle θ₂ spreads in accordance with the shape of the roughness. However, the emission angle θ₂, when averaged, becomes close to the incident angle θ₁ in many cases because a diameter of the light flux 12 is comparatively large.

If the incident light 6 is vertically entered with respect to the surface 13 of the target object 9, an optical axis is shifted in the vertical direction with respect to the surface 13 of the target object 9 in some cases because an error occurs in an angle adjustment, and the angle adjustment error is generally about from several degrees or lower to 10 degrees.

As illustrated in FIG. 2B, for example, on the lower surface of the light detector 2 to which the direct reflection light 5 is projected, when a center position of the light flux 12 of the direct reflection light 5 matches an A point on an edge in the inner circumference of the photo receiving element 3, about 50% of the direct reflection light 5 passes through the region 4. This position is set as a limit position where a shift of the optical axis of the direct reflection light 5 is allowable. The thickness of the light detector 2 is thin, and thus if the thickness of the light detector 2 is ignored, a condition that the light flux 12 enters the region 4 interior than the position of the A point is obtained as a relational expression of d₁/2S₁(tan θ₁+tan θ₂). The apparatus may be configured by determining the parameters such as the size d₁ of the region 4, the distance S₁, and the angles θ₁, θ₂ to satisfy the relational expression.

The abovementioned relational expression becomes d₁≧4S₁ tan θ₁ because θ₁=θ₂ is satisfied in many cases. Similar to the abovementioned condition, it can be understood from these expressions that the rate of the direct reflection light 5 that passes through the region 4 is raised by methods including making the size d₁ of the region 4 be larger, making the distance S₁ from the photo receiving element 3 to the surface 13 of the target object 9 be smaller, or making the incident angle θ₁ closer to 0 (normal incidence), thereby making it possible to obtain the high signal-to-noise ratio.

For example, when 10° is set to a maximum value for θ₁, for example, d₁≧0.705S₁ is obtained. For example, d₁≧7.05 mm when S₁=10 mm and d₁3.5 mm when S₁=5 mm are obtained. In this manner, the size d₁ of the region 4 may preferably be determined in accordance with the measurement distance and the angle adjustment error.

Moreover, if the structure of the optical sensing apparatus 100 is determined as the above, the incident angle θ₁ of the incident light 6 to the target object 9 is derived as θ₁≦tan⁻¹(d₁/(4S₁)) from the abovementioned expression. In other words, the laser chip 10 may irradiate the target object 9 with the incident light 6 such that the incident angle θ₁ of the incident light 6 to the target object 9 satisfies θ₁≦tan⁻¹(d₁/(4S₁)) when the size of the region 4 is denoted by d₁ and the distance from the surface of the photo receiving element 3 that is opposed to the target object 9 to the center of the light flux of the incident light 6 on the surface of the target object 9 is denoted by S₁. Accordingly, the optical sensing method according to the present embodiment may include a step of emitting the incident light 6 towards the target object 9 such that the incident angle θ₁ satisfies the relational expression, and a step of detecting the reflection scattered light 8 from the inside of the target object 9. This improves the signal-to-noise ratio of the detection for the reflection scattered light 8.

The light detector 2 may be configured such that the direct reflection light 5 hardly enters the photo receiving element 3. When a spot diameter of the direct reflection light 5 on the surface of the photo receiving element 3 is denoted by w, a condition that the entire light flux 12 enters the region 4 is expressed by a relational expression of d₁/2≧w/2+S₁(tan θ₁+tan θ₂).

The abovementioned relational expression becomes d₁≧w+4S₁ tan θ₁ because θ₁=θ₂ is satisfied in many cases. The optical sensing apparatus 100 may be configured by determining parameters to satisfy the relational expression.

Moreover, the incident angle θ₁ of the incident light 6 to the target object 9 is derived as θ₁≦tan⁻¹((d₁−w)/(4S₁)) from the abovementioned expression. In other words, when a spot diameter of the direct reflection light 5 on the surface of the photo receiving element 3 is denoted by w, an incident angle θ₁ of the incident light 6 to the target object 9 satisfies θ₁≦tan⁻¹((d₁−w)/(4S₁)). Therefore, the optical sensing method according to the present embodiment may include a step of emitting the incident light 6 towards the target object 9 such that the incident angle θ₁ satisfies the relational expression, and a step of detecting the reflection scattered light 8 from the inside of the target object 9. This further improves the signal-to-noise ratio of the detection for the reflection scattered light 8.

Note that, a case where the optical axis of the incident light 6 is already inclined when being emitted from the laser chip 10 is explained in the abovementioned embodiment. However, the embodiment is not limited to this, and applies to a case where the incident light 6 to be emitted from the laser chip 10 is not inclined, but the laser chip 10 itself is inclined with respect to the surface of the target object 9.

As described above, the optical sensing apparatus 100 according to the present embodiment is provided with a light source that emits light with which the target object 9, is irradiated, and the light detector 2 that is disposed on an optical axis from the light source to the target object. The light detector 2 is disposed on the optical axis from the light source to the target object 9. The light detector 2 includes the region 4 through which the incident light 6 emitted from the light source passes, and the photo receiving element 3 that receives the reflection scattered light 8 from the inside of the target object 9, the reflection scattered light 8 being generated in such a manner that the incident light 6 having passed through the region 4 is projected to the target object 9, and converts the received light into electric signals.

This causes the incident light 6 to enter in the approximate vertical direction with respect to the surface 13 of the target object 9 through the region 4 of the light detector 2, thereby allowing the incident light 6 to deeply enter the inside of the target object. Accordingly, the reflection scattered light 8 from the inside of the target object 9 can be detected with high accuracy.

Moreover, the direct reflection light 5 from the surface of the target object 9 is caused to pass through the region 4 of the light detector 2 (passed in a reverse direction relative to the incident light), thereby making it possible to suppress the direct reflection light 5 from being projected to the photo receiving element 3. Therefore, the reflection scattered light 8 from the inside of the target object 9 can be mainly detected.

### Second Embodiment

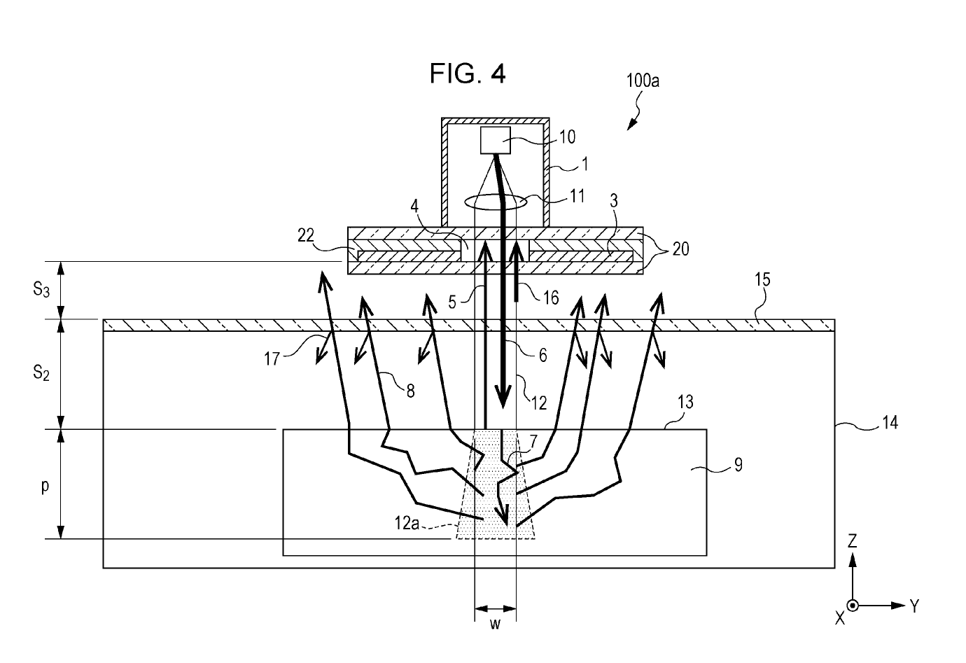

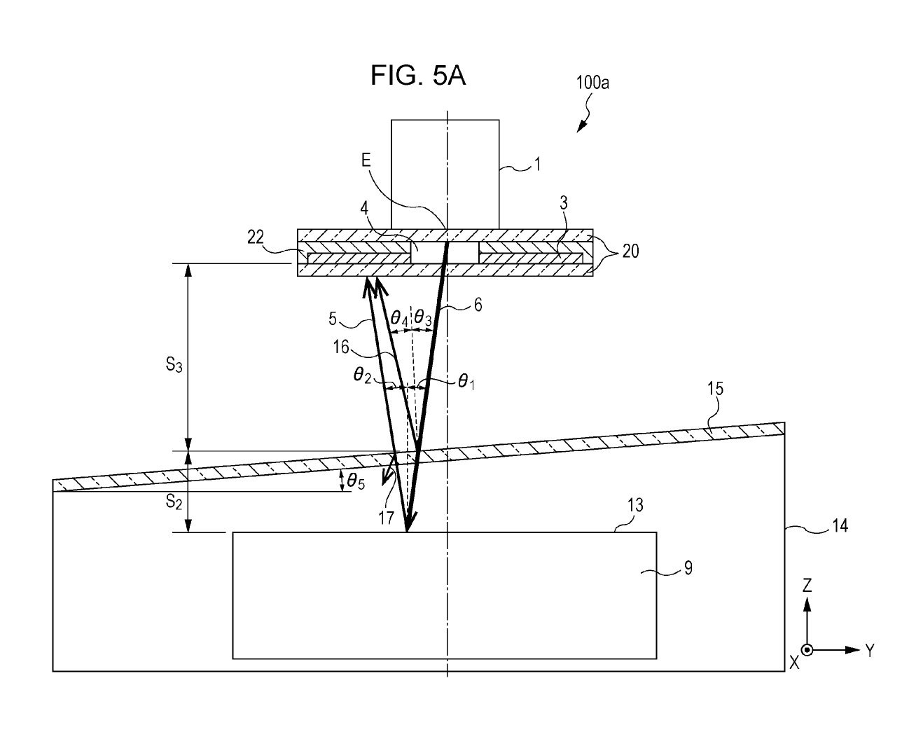

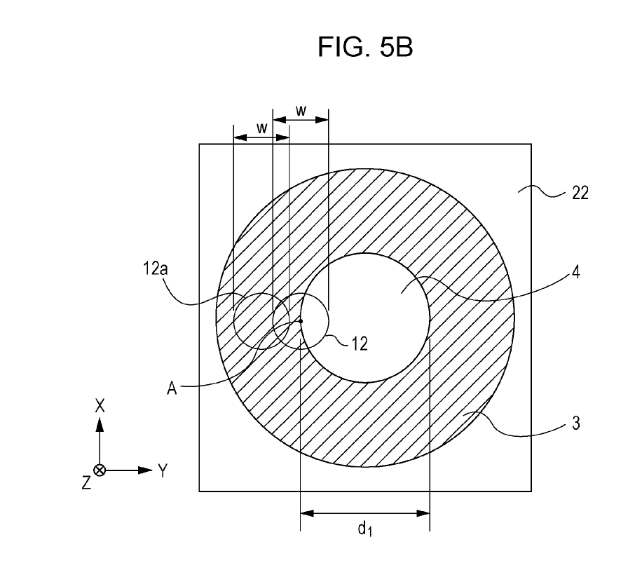

Next, as for an optical sensing apparatus according to a second embodiment, different points from the optical sensing apparatus in the abovementioned first embodiment will be mainly described using FIGS. 4, 5A, and 5B. FIG. 4 is an explanation view illustrating a configuration of the optical sensing apparatus according to the present embodiment, and a situation where the optical sensing apparatus irradiates a target object with light from a light source, and detects light. FIG. 5A is an explanation view illustrating, when an optical axis of incident light and an installation angle of a transparent lid are inclined from reference values with respect to the surface of the target object, light beams of the incident light, direct reflection light from the transparent lid, and direct reflection light from the target object, in the optical sensing apparatus according to the present embodiment. FIG. 5B is an explanation view illustrating, when an optical axis of incident light and an installation angle of a transparent lid are inclined from reference values with respect to the surface of the target object, a position of a light flux of the direct reflection light from the transparent lid to the photo receiving element side of the light detector, and a position of a light flux of the direct reflection light from the target object, in the optical sensing apparatus according to the present embodiment.

Note that, in FIG. 5A, for easy explanation, illustration of internal scattered light and reflection scattered light from the target object is omitted, and light beams indicating the progression of the incident light and the two direct reflection lights are mainly illustrated.

An optical sensing apparatus 100a according to the present embodiment is different from the optical sensing apparatus 100 according to the first embodiment in a configuration of a light detector 22, and in that the target object 9 is contained in a container 14 that is provided with a transparent lid 15. The target object 9 is specifically food, such as perishable food, that is contained in a container through which consumers can see the food. The transparent lid 15 may be a transparent lid made of a resin, or may be a transparent film made of a resin such as so-called plastic wrap. Note that, a transparent lid and a transparent film are collectively called the transparent lid 15 in the present embodiment.

The light detector 22 has such a structure that the photo receiving element 3 is shielded by and interposed between transparent substrates 20 that are made of a glass or a resin, as illustrated in FIG. 4. Such a structure can protect the photo receiving element 3 against an environment such as the high humidity, and thereby can improve the environment resistance of the optical sensing apparatus 100a.

In FIG. 4, a distance in the Z direction from the photo receiving element 3 to a center position of the transparent lid 15 is denoted by S₃, and a distance in the Z direction from the center position of the transparent lid 15 to the surface 13 of the target object 9 is denoted by S₂.

As illustrated in FIG. 4, the incident light 6 that is emitted in the −Z direction from the laser chip 10 is reflected on a surface of the transparent lid 15 by the reflectance of about 8 to 10%, for example, because the refractive index of the transparent lid 15 is n=1.5 to 1.6, and reflection occurs on both faces at both sides, and thereby becomes a direct reflection light 16. Moreover, remaining light passes through the transparent lid 15, and substantially vertically is incident into the target object 9. The light incident into the inside of the target object 9 is scattered by the inside tissue to become the internal scattered light 7. The reflection scattered light 8 that comes out from the inside of the target object 9 is again directly reflected on the transparent lid 15 by the reflectance of about 8 to 10%, for example, to become direct reflection light 17 from the transparent lid 15. Remaining light except the direct reflection light 17 passes through the transparent lid 15 and the transparent substrate 20, and is detected by the photo receiving element 3 of the light detector 22.

Meanwhile, the reflectance at the surface 13 of the target object depends on the refractive index thereof, and is a half of or less than the reflectance of the transparent lid 15 in many cases if the target object is perishable food, for example.

In the configuration of the optical sensing apparatus 100a according to the present embodiment, the quantity of light of the direct reflection light 16 that is reflected from the transparent lid 15 tends to generally become further larger (for example, two times or more) than the quantity of light of the direct reflection light 5 from the surface 13 of the target object 9. However, both of the direct reflection light 5 and the direct reflection light 16 are mainly incident into the region 4, and thus the reflection scattered light 8 is mainly incident into the photo receiving element 3. Accordingly, the optical sensing apparatus 100a can acquire a signal with a high signal-to-noise ratio.

Next, a case where the optical axis of the incident light 6 and an installation angle of the transparent lid 15 are inclined from reference values, with respect to the surface 13 of the target object 9, in the optical sensing apparatus 100a according to the present embodiment will be described. The reference value for the incident angle of the incident light 6 is a normal incidence (incident angle θ₁=0) with respect to the surface 13 of the target object 9, and the reference value for the installation angle of the transparent lid 15 is set to that of the case where the transparent lid 15 is in parallel with the surface 13 (θ₅=0).

As illustrated in FIG. 5A, the incident light 6 is incident on the surface 13 of the target object 9 in an inclined direction at an incident angle θ₁, and is incident into the transparent lid 15 at an installation angle θ₅ before being projected to the surface 13 of the target object 9. The installation angle θ₅ is an inclination angle of the transparent lid 15 with respect to the surface of the target object 9, and as illustrated in FIG. 5A, a positive sign is set when the transparent lid 15 is raised on the right side. An incident angle in that case is denoted by θ₃(=θ₁+θ₅) with respect to the normal to the transparent lid 15. An emission angle is denoted by θ₄, and θ₃=θ₄ is obtained because the transparent lid 15 can be approximated to be flat. The angles θ₁ and θ₅ are realistically about several degrees to 10 degrees in many cases.

As illustrated in FIG. 5B, for example, on a lower surface of the light detector 22, a spot center of a light flux 12 of the direct reflection light 16 from the transparent lid 15 matches the A point on the edge in the inner circumference of the photo receiving element 3. When the spot center of the light flux 12 is positioned on the inner circumference of the photo receiving element 3, approximately 50% of the direct reflection light 5 from the target object 9 and approximately 50% of the direct reflection light 16 from the transparent lid 15 pass through the region 4. Accordingly, a condition that the light flux 12 may enter the region 4 from the present position is approximated to d₁/2S₃(tan θ₃+tan θ₄)=2S₃ tan θ₃=2S₃ tan(θ₁+θ₅). The apparatus may be configured by determining the parameters such as the size of the region 4 (diameter) d₁, a distance S₃ from the surface of the photo receiving element 3 opposed to the target object 9 to the center of the light flux of the incident light 6 on the surface of the transparent lid 15, and the angles θ₁, θ₅ may be configured to satisfy this expression.

As can be understood from the abovementioned expression, the rate of the direct reflection light 16 from the transparent lid 15 that passes through the region 4 is raised by methods including making the size d₁ of the region 4 larger, making the distance S₃ in the Z direction from the photo receiving element 3 to the transparent lid 15 smaller, or making the angles θ₁, θ₅ closer to 0 (normal incidence), thereby making it possible to obtain the high signal-to-noise ratio.

Moreover, the laser chip 10 that is a light source may irradiate the target object 9 with the incident light 6 such that the incident angle θ₁ of the incident light 6 to the target object 9 satisfies θ₁≦tan⁻¹(d₁/(4S₃))−θ₅ when a size of the region 4 is denoted by d₁, a distance from the surface of the photo receiving element 3 opposed to the target object 9 to the center of the light flux of the substantially parallel light 6 on the surface of the transparent lid 15 is denoted by S₃, and an inclination angle of the transparent lid 15 with respect to the surface of the target object 9 is denoted by θ₅. Therefore, the optical sensing method according to the present embodiment may include a step of emitting the incident light 6 towards the target object 9 by the light source such that the incident angle θ₁ of the incident light 6 to the target object 9 satisfies the relational expression, and a step of detecting the reflection scattered light 8 from the inside of the target object 9 by the light detector 22.

In addition, the direct reflection light 16 from the transparent lid 15 may hardly enter the photo receiving element 3 of the light detector 22. When a spot diameter of the direct reflection light 16 on the surface of the photo receiving element 3 is denoted by w, a condition that the entire light flux 12 enters the region 4 is expressed by d₁/2≧w/2+S₃(tan θ₁+tan θ₂).

The abovementioned relational expression becomes d₁≧w+4S₃ tan θ₁ because θ₁=θ₂ is satisfied in many cases.

The apparatus may be configured by determining parameters to satisfy the relational expression. The signal-to-noise ratio of the detection for the reflection scattered light 8 from the inside of the target object 9 is further improved.

Moreover, the optical sensing method according to the present embodiment may include a step of, when a spot diameter of the direct reflection light 16 that is the incident light 6 being reflected on the surface of the transparent lid 15, on the surface of the photo receiving element 3, is denoted by w, emitting the incident light 6 towards the target object 9 such that the incident angle θ₁ of the incident light 6 to the target object 9 satisfies θ₁≦tan⁻¹((d₁−w)/(4S₃))−θ₅, by the light source, and a step of detecting the reflection scattered light 8 from the inside of the target object 9 by the light detector 22.

Next, the direct reflection light 5 from the surface 13 of the target object 9 is examined. For example, when, in the lower surface of the light detector 22, the spot center of the light flux 12 of the direct reflection light 5 from the surface 13 of the target object 9 is positioned on the A point, in other words, on an edge in the inner circumference of the light receiving element 3, approximately 50% of the direct reflection light 5 passes through the region 4. Accordingly, a condition that the light flux 12 may be positioned in the region 4 interior than the present position is approximated to d₁/2≧(S₂+S₃)(tan θ₁+tan θ₂). The apparatus may be configured by determining the parameters such as the size d₁ of the region 4, the distances S₂, S₃, and the angles θ₁, θ₂ to satisfy the relational expression. Note that, when the surface 13 is flat, d₁≧4(S₂+S₃)tan θ₁ is obtained.

From the abovementioned expression, it can be understood that the rate of the direct reflection light 5 that passes through the region 4 is raised by methods including making the size d₁ of the region 4 larger, making the distance S₂+S₃ in the Z direction from the photo receiving element 3 to the surface 13 of the target object 9 smaller, or making the incident angle θ₁ closer to 0 (normal incidence), thereby making it possible to obtain the high signal-to-noise ratio.

The optical sensing apparatus 100a according to the present embodiment may be constructed to simultaneously satisfy the abovementioned two expressions so that the direct reflection light 5 and the direct reflection light 16 can be prevented from entering into the photo receiving element 3 as much as possible.

As described above, the optical sensing apparatus 100a according to the present embodiment causes not only the direct reflection light 5 from the target object 9 but also the direct reflection light 16 from the transparent lid 15 to pass though the region 4 of the light detector 22 also with respect to the target object 9 that is covered by the transparent lid 15, so that the photo receiving element 3 can detect the reflection scattered light 8 from the inside of the target object 9 with the excellent signal-to-noise ratio.

Moreover, the light detector 22 may have such a structure that the photo receiving element 3 is held between and shielded by the transparent substrates 20 that are made of a glass or a resin. Such a structure can protect the photo receiving element 3 against an environment such as the high humidity, and thereby can improve the environment resistance of the optical sensing apparatus 100a.

### Third Embodiment

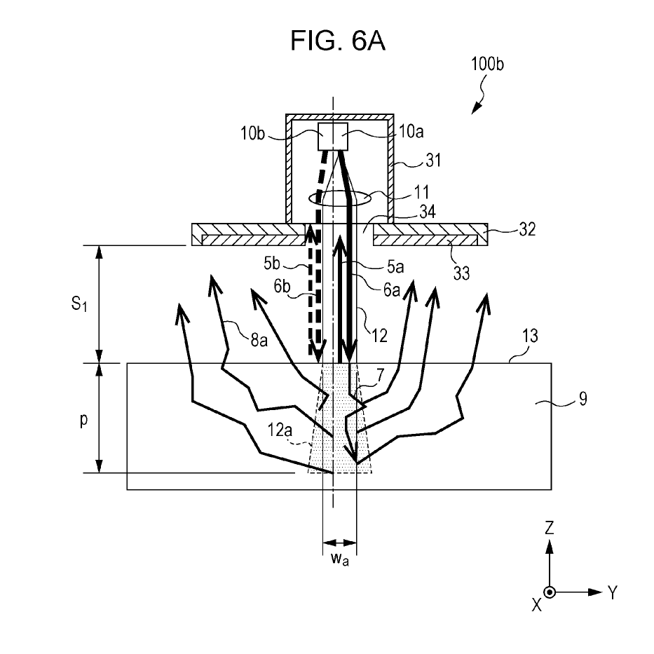

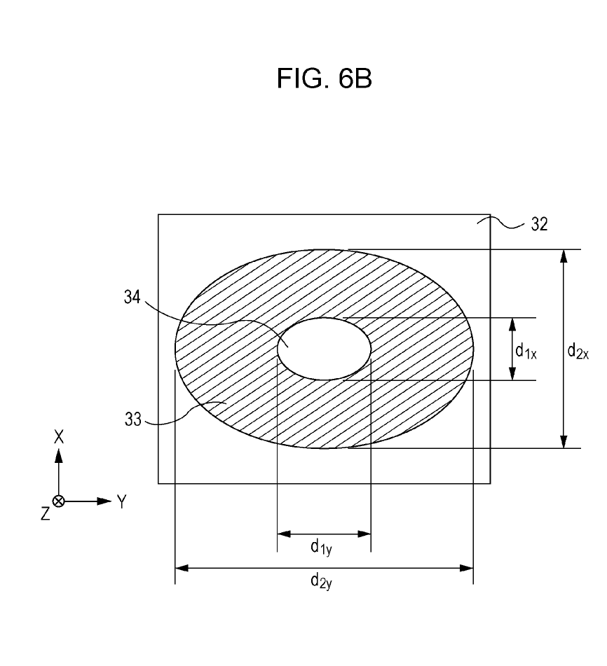

Next, as for an optical sensing apparatus according to a third embodiment, different points from the optical sensing apparatus in the abovementioned first embodiment will be mainly described using FIGS. 6A and 6B. FIG. 6A is an explanation view illustrating a configuration of the optical sensing apparatus according to the present embodiment, and a situation where the optical sensing apparatus irradiates a target object with light from a light source, and detects light, and FIG. 6B is a configuration view of a photo receiving element side of a light detector in the optical sensing apparatus according to the present embodiment.

An optical sensing apparatus 100b according to the present embodiment is different from the optical sensing apparatus 100 in the first embodiment in that the optical sensing apparatus 100b is provided with a laser chip 10a that emits light of a first wavelength and a laser chip 10b that emits light of a second wavelength as light sources, irradiates the target object 9 with light of each wavelength separately to receive reflection scattered light 8a and converts it into an electric signal, and calculates those converted signals, thereby obtaining information.

As illustrated in FIG. 6A, the two laser chips 10a, 10b are disposed in a housing 31 by being aligned in the Y direction. The optical sensing apparatus 100b according to the present embodiment can obtain living body information by irradiating a living body, for example, with light of different wavelengths. As one example, the optical sensing apparatus 100b can obtain living body information such as the oxygen saturation in blood by utilizing a fact that oxidized (or oxygenated) hemoglobin and deoxidized (or deoxygenated) hemoglobin absorb light of different wavelengths.

Specifically, in two wavelengths of λ₁=660 nm and λ₂=830 nm, the oxidized hemoglobin and the deoxidized hemoglobin have the different absorptance. Accordingly, electric signals that are obtained in the respective wavelengths are calculated to make it possible to measure the oxygen saturation in blood when the target object 9 is skin of a living body, for example.

Moreover, when the target object 9 is a forehead region of a head of the living body, a changing amount of a cerebral blood flow in a frontal lobe, and changing amounts of concentration of the oxidized hemoglobin and the deoxidized hemoglobin can be measured, so that sensing of information such as an emotion is possible. For example, in a concentrated state, increase in the blood flow in the brain, increase in the amount of the oxidized hemoglobin, or the like occurs. In particular, when the target object 9 is a head, the large attenuation of light in bones of skull results in the weak intensity of the reflection scattered light 8a from the inside (for example, 10⁻³ to 10⁻⁶ times the incident power), and large noise sources of direct reflection light 5a, 5b from the surface 13. However, the optical sensing apparatus 100b according to the present embodiment can reduce the influence by the direct reflection light 5a, 5b from the surface 13.

Various combinations of wavelengths are possible. For example, the absorptance of the oxidized hemoglobin and the deoxidized hemoglobin become the same at a wavelength of 805 nm, so that a combination of a wavelength less than 805 nm and a wavelength more than 805 nm may be used. Moreover, three wavelengths of a wavelength of 805 nm in addition to the two wavelengths can be also used. The calculation in the processor can be simplified in the case of the three wavelengths.

Moreover, as illustrated in FIG. 6B, in a light detector 32, a region 34 has an elliptic shape having a major axis in a direction (Y direction) in which the laser chips 10a, 10b that are light sources are arranged. The region 34 is formed in such an elliptic shape to allow spaces between light fluxes of incident light 6a, 6b and a photo receiving element 33 to be the same both in the X direction and the Y direction. With this, the direct reflection light 5a, 5b due to the projection of light from the laser chips 10a, 10b pass through the region 34, so that degradation of the signal-to-noise ratio of an electric signal that is generated from the light received by the photo receiving element 33 can be suppressed. Moreover, the photo receiving element 33 also may have an elliptic shape having a major axis in the Y direction.

### (Modification Example)

When the target object 9 is a forehead region of a head of the living body, as described above, the intensity of the reflection scattered light 8 is weak. Meanwhile, the direct reflection light 5a, 5b from the forehead surface and the skin of the forehead includes scatter components. Thus, some of the components of the direct reflection light 5a, 5b enter the photo receiving element 33. With this, when the target object 9 is a forehead region of the head of the living body, the signal-to-noise ratio tends to be degraded.

An optical sensing apparatus according to a modification example in the present embodiment is further provided with a drive circuit (not illustrated) that causes the laser chips 10a, 10b to emit light in a pulse shape. The drive circuit drives the laser chips 10a, 10b such that the laser chips 10a, 10b alternately emit pulse light having a pulse width, for example, in a range about from 100 picoseconds to several ten nanoseconds.

The incident light 6a, 6b is made to be pulse light to remove electric signals caused by the direct reflection light 5a, 5b from the electric signals acquired by the photo receiving element 33, by utilizing a time difference between times when the direct reflection light 5a, 5b and the reflection scattered light 8a from the inside of the target object 9 reach the photo receiving element 33. This can improve the signal-to-noise ratio.

For example, a case where a cerebral blood flow in a frontal lobe is detected is specifically described. The reflection scattered light 8a including information on the blood flow in the brain is generated in such a manner that light entering into a brain is reflected and scatter inside the brain, and thus has a light path difference with the direct reflection light 5a, 5b. Therefore, a time when the reflection scattered light 8a reaches the photo receiving element 33 is delayed by typically about 4 nanoseconds, for example, compared with the direct reflection light 5a, 5b. Therefore, an electric signal that is acquired, for example, 4 nanoseconds later after the direct reflection light 5a, 5b reaches the photo receiving element 33, is taken out from the photo receiving element 33, and components of the direct reflection light 5a, 5b are reduced from the acquired electric signal, thereby making it possible to improve the signal-to-noise ratio.

The pulse width of the pulse light may be 1 nanosecond or more. This can allow the simple configuration of the drive circuit that drives the laser chips 10a, 10b. The pulse width of the pulse light may be 20 nanoseconds or less. This can easily isolate the direct reflection light 5a, 5b from the reflection scattered light 8a.

### Fourth Embodiment

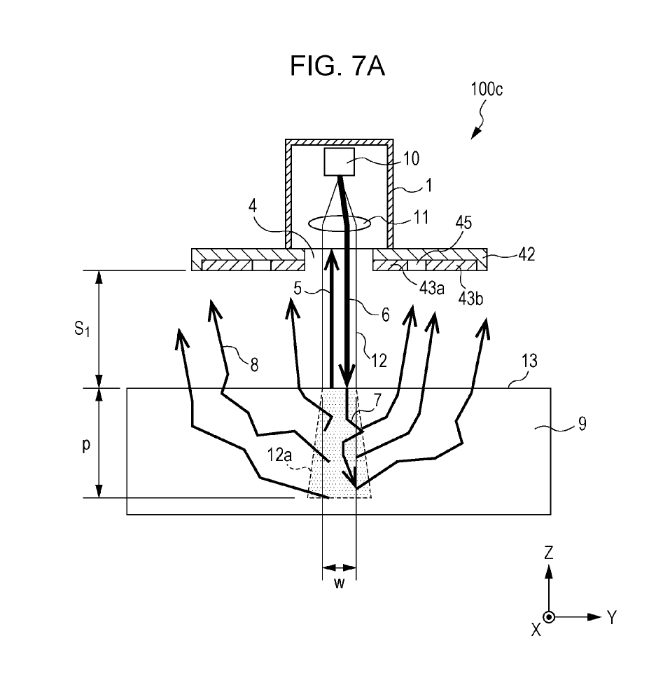

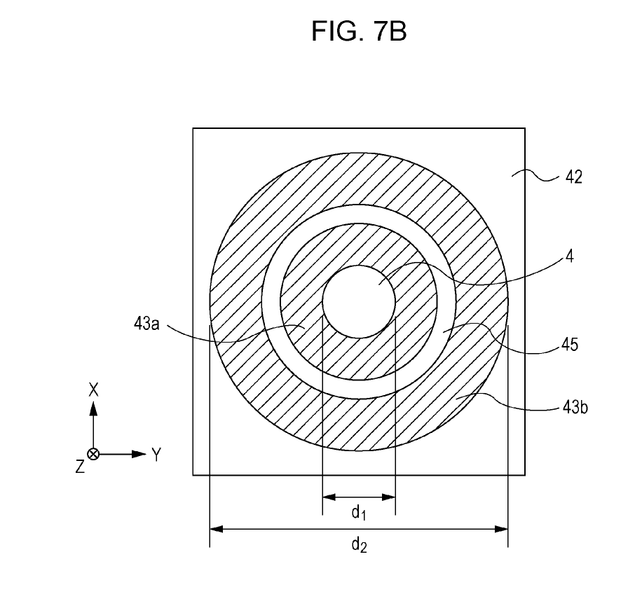

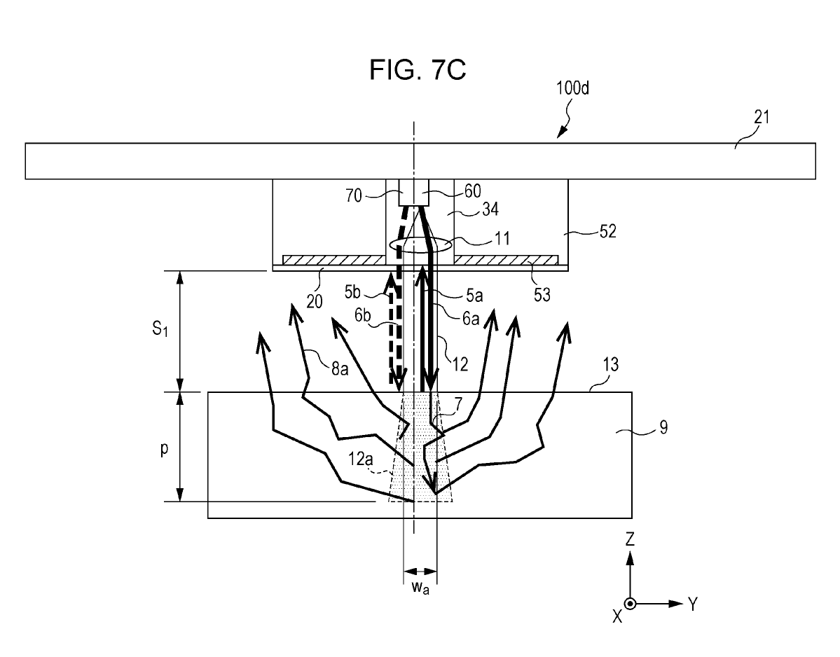

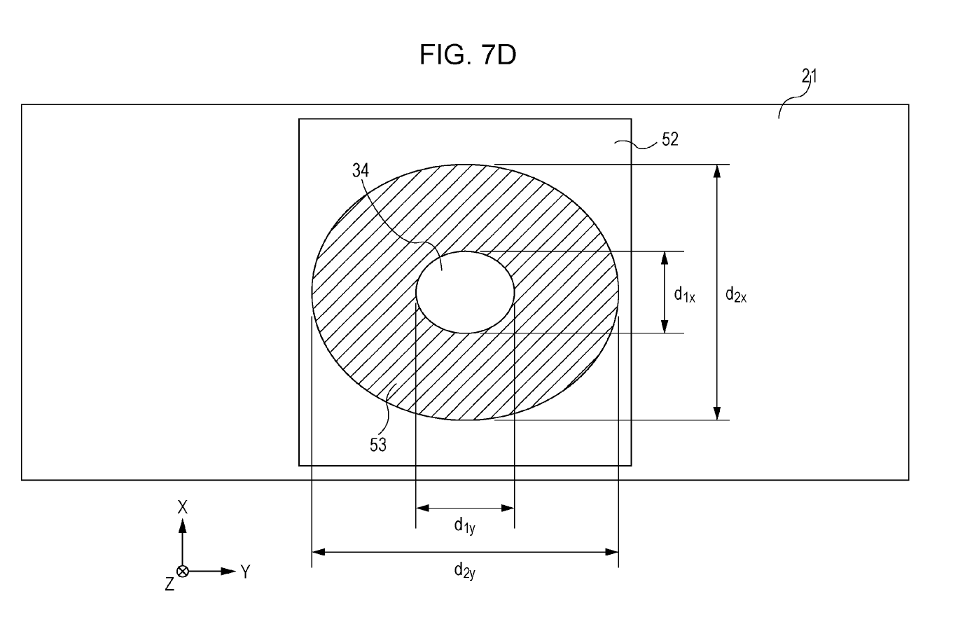

Next, as for an optical sensing apparatus according to a fourth embodiment, different points from the optical sensing apparatus in the abovementioned first embodiment will be mainly described using FIGS. 7A and 7B. FIG. 7A is an explanation view illustrating a configuration of an optical sensing apparatus 100c according to the present embodiment, and a situation where the optical sensing apparatus irradiates a target object with light from a light source, and detects light. FIG. 7B is a configuration view illustrating a configuration of a light detector on the photo receiving element side in the optical sensing apparatus according to the present embodiment.

The optical sensing apparatus 100c according to the present embodiment is different from the optical sensing apparatus 100 according to the first embodiment in that a light detector 42 according to the present embodiment includes multiple photo receiving elements 43a, 43b.

As illustrated in FIGS. 7A and 7B, the light detector 42 is provided with the region 4 through which the incident light 6 passes at a center portion thereof, similar to the light detector 2 illustrated in the first embodiment. Further, the photo receiving element includes the multiple photo receiving elements 43a, 43b that are divided in a radial direction of the region 4 in the surrounding of the region 4. Moreover, the light detector 42 further includes a space 45 between the photo receiving elements 43a, 43b. The photo receiving elements 43a, 43b are electrically insulated by the space 45.

In other words, the light detector 42 is provided with the ring-like (concentrically) photo receiving element 43a in the surrounding that surrounds the region 4. Moreover, the ring-like space 45 that is a region having no light receiving sensitivity is provided in the surrounding that surrounds the photo receiving element 43a. In addition, the photo receiving element 43b is provided in the surrounding of the space 45. As described above, the light detector 42 includes the two photo receiving elements 43a, 43b that are electrically insulated by the space 45. The light signals that are received by the photo receiving elements 43a, 43b are calculated as electric signals.

The reflection scattered light 8 comes out from the inside of the target object 9 in a ring-like shape at a position on the surface 13 having a radius substantially the same as the depth that light enters, so that the light detector 42 can detect information on a plural number of depth positions by using the multiple photo receiving elements that are electrically insulated to one another. For example, the photo receiving element 43a having a small radius is capable of obtaining information on the depth to the extent of the small radius, and the photo receiving element 43b having a large radius is capable of obtaining information on the depth to the extent of the large radius.

Moreover, a processor (not illustrated) performs calculation such as subtraction and addition processes for the above information, thereby making it possible to further remove noise components and improve the signal-to-noise ratio. For example, when the target object 9 is a living body, the information acquired by the photo receiving element 43a having a small radius is multiplied by a suitable proportionality coefficient, and the multiplication result is subtracted from the information acquired by the photo receiving element 43b having a large radius, thereby making it possible to reduce the noise components due to the surface blood flow.

Note that, in the abovementioned embodiment, the photo receiving element is divided into two regions, that is, the photo receiving elements 43a, 43b. However, the photo receiving element may be divided into three or more regions. In that case, a configuration in which multiple ring-like spaces 45 having different radii are concentrically disposed is applied, thereby making it possible to obtain information on the depth positions more finely.

Even in a case where the photo receiving element is divided into three or more regions, similar to a case where the photo receiving element is divided into two regions, the processor calculates information from the multiple photo receiving elements, thereby making it possible to improve the signal-to-noise ratio. (Fifth Embodiment)

Next, as for an optical sensing apparatus according to a fifth embodiment, different points from the optical sensing apparatus in the abovementioned third embodiment will be mainly described using FIGS. 7C and 7D. FIG. 7C is an explanation view illustrating a configuration of the optical sensing apparatus according to the present embodiment, and a situation where the optical sensing apparatus irradiates a target object with light from a light source, and detects light, and FIG. 7D is a configuration view of a light detector on the photo receiving element side in the optical sensing apparatus according to the present embodiment.

An optical sensing apparatus 100d according to the present embodiment is different from the optical sensing apparatus 100b in the third embodiment in that a light detector 52, and surface-emitting laser chips 60, 70 that are light sources are disposed on a flexible substrate 21 that can be bent gently.

Providing the surface-emitting laser chips 60, 70 and the light detector 52 on the flexible substrate 21 makes it possible to fabricate the optical sensing apparatus 100d that can be bent gently. For example, the optical sensing apparatus 100d is attached to a cloth, thereby making it possible to use the optical sensing apparatus 100d as a wear-comfortable wearable type living body sensing apparatus.

For example, the optical sensing apparatus 100d is attached to a belt-like cloth that can be mounted to a head to obtain a wearable type optical sensing apparatus that is capable of sensing a blood flow in the brain. The belt-like cloth is mounted to the head such that the flexible substrate 21 of the optical sensing apparatus 100d comes into contact with or comes close in about several millimeters to a forehead, thereby making it possible to perform the sensing of the blood flow in the brain.

In the present embodiment, the multiple surface-emitting laser chips 60, 70 as light sources are arranged in the Y direction on the flexible substrate 21 that is made of, for example, polyimide, and provided with electric wiring. The use of the surface-emitting laser chip allows the light source to be thinner, in other words, to be reduced in size in the Z direction. Moreover, a diffractive optical lens or a Fresnel lens is employed as the collimator lens 11, thereby making the thinner collimator lens 11.

A heat sink film having an electrical insulation property, for example, a film in which a graphite film and an oxide film such as SiO₂ are combined may be provided between the flexible substrate 21 and the surface-emitting laser chips 60, 70. This allows easy heat radiation of the optical sensing apparatus 100d.

A photo receiving element 53 is formed of a thin-film material, for example, amorphous Si, poly Si, or organic EL material, thereby making it possible to obtain the photo receiving element with the flexibility.

Moreover, the electric wiring is formed with a metal thin film on the flexible substrate 21, and a thin battery such as a polymer battery is also disposed thereon, thereby making it possible to fabricate a wearable type optical sensing apparatus that can be driven by the battery.

According to the first to fifth embodiments described above, it is possible to fabricate the optical sensing apparatuses capable of reducing direct reflection light from the surface of the target object, and direct reflection light from plastic wrap or the transparent lid if being present, and excellently detect reflection scattered light from the inside thereof where much information is present.

Note that, the present disclosure is not limited to these embodiments, but an optical sensing apparatus and an optical sensing method in which the configurations of the optical sensing apparatuses in the respective embodiments are combined are included in the present disclosure, and can exhibit the similar effect.

For example, the abovementioned light detector may have such a structure that the photo receiving element is shielded by and interposed between the transparent substrates that are made of a glass or a resin. Such a structure can protect the photo receiving element against an environment such as the high humidity, and thereby can improve the environment resistance of the optical sensing apparatus.

Moreover, when a photo receiving element is divided into multiple regions, the photo receiving element may be radially divided, in the surrounding of a region through which incident light passes, in a circumferential direction of the region, or may be divided in a ring-like shape in a radial direction of the region, in the surrounding of the region.

When the photo receiving element is radially divided, the number of regions to be divided is not limited to the abovementioned number, but may be changed as appropriate. In this process, the number of spaces between adjacent two photo receiving elements may be changed as appropriate. The increase in the number of photo receiving elements makes it possible to obtain information on a living body in in-plane directions more finely.

When the photo receiving element is divided into a ring-like shape, as described above, the number of regions to be divided is not limited to two regions, but the photo receiving element may be divided into three or more regions. The increase in the number of photo receiving elements makes it possible to obtain information on a living body at depth positions more finely.

What is claimed is:

1. An optical sensing apparatus comprising:
at least one light source that, in operation, emits light with which a target object is irradiated; and
a light detector which includes
a region in which at least a part of an optical axis from the at least one light source to the target object is located, and through which the light emitted from the at least one light source passes, and
at least one photo receiving element that, in operation, receives reflection scattered light from an inside of the target object, the reflection scattered light being generated due to the irradiation of the target object with the light having passed through the region, and converts the reflection scattered light into an electric signal.

2. The optical sensing apparatus according to claim 1, wherein the at least one photo receiving element is in contact with the region.

3. The optical sensing apparatus according to claim 1, wherein the at least one photo receiving element surrounds the region in plan view.

4. The optical sensing apparatus according to claim 1, wherein a spread angle of the light having passed through the region is within ±3° in a total angle.

5. The optical sensing apparatus according to claim 1, further comprising a collimator lens disposed on the optical axis.

6. The optical sensing apparatus according to claim 1, wherein a beam diameter of the light having passed through the region is from 200 μm to 20 mm, both inclusive.

7. The optical sensing apparatus according to claim 1, wherein the at least one light source comprises a first light source that, in operation, emits light of a first wavelength, and a second light source that, in operation, emits light of a second wavelength.

8. The optical sensing apparatus according to claim 7, wherein
in plan view,
the first light source and the second light source are disposed along a first direction, and
the region has an elliptic shape having a major axis in the first direction.

9. The optical sensing apparatus according to claim 1, wherein
the at least one photo receiving element comprises photo receiving elements, and
the photo receiving elements are disposed in a surrounding area of the region in a circumferential direction of the region with spaces that electrically insulate the photo receiving elements from one another.

10. The optical sensing apparatus according to claim 1, wherein
the at least one photo receiving element comprises photo receiving elements, and
the photo receiving elements are disposed in a surrounding area of the region in a radial direction of the region with a space that electrically insulates the photo receiving elements from one another.

11. The optical sensing apparatus according to claim 1, wherein the light emitted by the at least one light source is pulse light.

12. The optical sensing apparatus according to claim 1, further comprising a flexible substrate, wherein
the at least one light source and the light detector are disposed on the flexible substrate.

13. The optical sensing apparatus according to claim 1, further comprising a processor, wherein, in operation,
the processor performs computation of the electric signal to obtain information on the target object.
